SEQUENCE LISTING (8 sequences)

SEQ ID NO: 1 - protein, 616 aa, Artificial Sequence
  A. niger amylase with a carbohydrate binding module from A. kawachii
AEWRTQSIYFLLTDRFGRTDNSTTATCDTGDQIYCGGSWQGIINHLDYIQGMGFTAIWISPITEQLPQDTADGEAYHGYWQQKIYDVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVLEVEPDFFPGYQEAAGVYCVGEVDNGNPALDCPYQKVLDGVLNYPIYWQLLYAFESSSGSISNLYNMIKSVASDCSDPTLLGNFIENHDNPRFASYTSDYSQAKNVLSYIFLSDGIPIVYAGEEQHYSGGKVPYNREATWLSGYDTSAELYTWIATTNAIRKLAISADSAYITYANDAFYTDSNTIAMRKGTSGSQVITVLSNKGSSGSSYTLTLSGSGYTSGTKLIEAYTCTSVTVDSSGDIPVPMASGLPRVLLPASVVDSSSLCGGSGRTTTTTTAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGNGSGETVVDTWR

SEQ ID NO: 2 - protein, 558 aa, Artificial Sequence
  Rhizomucor pusillus amylase with linker and starch binding domain from A. rolfsii
SPLPQQQRYGKRATSDDWKSKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSAGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 3 - protein, 574 aa, Artificial Sequence
  Hybrid of Meripilus giganteus amylase with A. rolfsii starch binding domain
RPTVFDAGADAHSLHARAPSGSKDVIIQMFEWNWDSVAAECTNFIGPAGYGFVQVSPPQETIQGAQWWTDYQPVSYTLTGKRGDRSQFANMITTCHAAGVGVIVDTIWNHMAGVDSGTGTAGSSFTHYNYPGIYQNQDFHHCGLEPGDDIVNYDNAVEVQTCELVNLADLATDTEYVRGRLAQYGNDLLSLGADGLRLDASKHIPVGDIANILSRLSRSVYITQEVIFGAGEPITPNQYTGNGDVQEFRYTSALKDAFLSSGISNLQDFENRGWVPGSGANVFVVNHDTERNGASLNNNSPSNTYVTATIFSLAHPYGTPTILSSYDGFTNTDAGAPNNNVGTCSTSGGANGWLCQHRWTAIAGMVGFRNNVGSAALNNWQAPQSQQIAFGRGALGFVAINNADSAWSTTFTTSLPDGSYCDVISGKASGSSCTGSSFTVSGGKLTATVPARSAIAVHTGQKGSGGGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 4 - protein, 384 aa, A.aculeatus
  mat_peptide (1)..(384)
VGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 5 - protein, 347 aa, Trichoderma reesei
  mat_peptide (1)..(347)
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 6 - protein, 419 aa, Trichoderma sp.
  mat_peptide (1)..(419)
MNKPMSSLLLAATLLAGGSIAQQTVWGQCGGQGWSGPTSCVAGSACSTLNPYYAQCIPGATTMSTTTKPTSVSASTTRASATSSATPPPSSGLTRFAGVNIAGFDFGCGTDGTCVTSKVYPPLKNYAGTNNYPDGVGQMQHFVNDDKLTIFRLPVGWQYLVNNNLGGTLDSNNFGKYDQLVQACLSLGVYCIVDIHNYARWNGGIIGQGGPTNDQFTSLWSQLAQKYASQSKVWFGIMNEPHDVNINTWATTVQAVVTAIRNAGATSQFISLPGNDWQSAGAFISDGSAAALSQVKNPDGSTPNLIFDLHKYLDSDNSGTHADCVTNNVNDAFSPVATWLRQNNRQAILTETGGGNTQSCIQYLCQQFQYINQNSDVYLGYVGWGAGSFDSTYILTETPTGSGSSWTDTSLVSSCISRK

SEQ ID NO: 7 - protein, 459 aa, Trichoderma viride
  mat_peptide (1)..(459)
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVGITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 8 - protein, 232 aa, Trichoderma sp.
  mat_peptide (1)..(232)
MKFLQVLPALIPAALAQTSCDQWATFTGNGYTVSNNLWGASAGSGFGCVTAVSLSGGAHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

(19) United States
(12) Patent Application Publication
(10) Pub. No.: US 2009/0117630 A1
(43) Pub. Date: May 7, 2009

(54) FERMENTATION PRODUCT PROCESSES

(75) Inventors: Hans Sejr Olsen, Holte (DK); Sven Pedersen, Gentofte (DK)

Correspondence Address: NOVOZYMES NORTH AMERICA, INC., 500 FIFTH AVENUE, SUITE 1600, NEW YORK, NY, 10110, US

(73) Assignee: NOVOZYMES A/S, Bagsvaerd (DK)

(21) Appl. No.: 11/720,098

(22) PCT Filed: Dec. 16, 2005
(86) PCT No.: PCT/DK05/00800

§ 371 Date: May 24, 2007

(30) Foreign Application Priority Data
Dec. 22, 2004 (DK) PA 2004 01977

(51) Int. Cl. C12P 7/06; C12N 9/28; C12N 9/34; C12N 9/24

(52) U.S. Cl. 435/161; 435/202; 435/205; 435/200

(57) ABSTRACT

The present invention relates to processes for production of an ethanol product from granular starch

### FIELD OF THE INVENTION

The present invention relates to processes for production of a fermentation product from granular starch. The use of a new combination of enzymes facilitates processing of a high dry solids mash.

### BACKGROUND OF THE INVENTION

Processes for conversion of granular starch into fermentation products, e.g. ethanol products, are disclosed in WO 03/066826, WO 04/080923 and WO 04/081193. To make such processes profitable there is a need for new processes which enable processing of high dry solids mash. The object of the present invention is to provide such improved processes for conversion of granular starch into fermentation products.

### SUMMARY OF THE INVENTION

The present inventors have surprisingly discovered that the use of an acid alpha-amylase comprising a carbohydrate binding module (CBM) in a granular starch liquefaction and saccharification facilitates the processing of a high dry solids mash.

The present inventors have further surprisingly discovered that the use of a combination of an acid alpha-amylase comprising a carbohydrate binding module (CBM) with viscosity reducing enzymes, such as xylanase and beta-glucanase in a granular starch liquefaction and saccharification facilitates the processing of a high dry solids mash, even when the grit comprises dry milled barley, rye or wheat and other grain types with a high xylan and beta-glucan content. By the effect of the superior raw starch degrading ability of the acid alpha-amylase comprising a CBM and/or the action of the viscosity reducing enzymes the processing of higher dry solids mash, a higher ethanol yield and thus a higher productivity and throughput can be obtained.

The application of viscosity reducing enzymes such as beta-glucanase and xylanase in the process of the invention degrades glucan and xylan thereby reducing the viscosity of the mash. The reduced viscosity results in increased flow rates of the liquefied mash, thereby increasing the capacity of the production plants. Furthermore, the invention provides a simplified process wherein a separate viscosity reducing step can be omitted.

The effect on the distillation process of the prior hydrolysis of non-starch polysaccharides like arabinoxylan and beta-glucans is an overall increased capacity and better heat transfer and phase transfer.

The effect on the by-products, such as the distiller's dry grain, of the prior hydrolysis of the non-starch polysaccharides as well as a more complete hydrolysis of the starch polysaccharides is an overall improved feed conversion and better digestibility of the nutrients like minerals, protein, lipids and residual starch.

Accordingly the present invention provides methods for producing an ethanol product from granular starch without prior gelatinization of said starch. In a first aspect, the invention provides a process comprising the steps of, a) providing a slurry comprising water and granular starch, b) holding said slurry in the presence of i) an acid alpha-amylase comprising a carbohydrate binding module, and ii) a fermenting organism, to produce a fermentation product and, c) optionally recovering the fermentation product. The fermentation organism is preferably yeast and the fermentation product preferably ethanol.

In a second aspect the invention provides a composition comprising i) an acid alpha-amylase comprising a CBM and, ii) a xylanase, and/or iii) a beta-glucanase, and/or iv) a glucoamylase.

### DETAILED DESCRIPTION OF THE INVENTION

By the process of the invention granular starch can be hydrolysed to maltose, glucose or specialty syrups, either for use as sweeteners or as precursors for other saccharides such as fructose. Maltose and/or glucose may also be fermented to an ethanol product or other fermentation products, such as citric acid, monosodium glutamate, gluconic acid, sodium gluconate, calcium gluconate, potassium gluconate, glucono delta lactone, or sodium erythorbate, itaconic acid, lactic acid, gluconic acid; ketones; amino acids, glutamic acid (sodium monoglutaminate), penicillin, tetracyclin; enzymes; vitamins, such as riboflavin, B12, beta-carotene or hormones.

The term “ethanol product” means a product comprising ethanol, e.g. fuel ethanol, potable and industrial ethanol. However, the ethanol product may also be a beer, which beer may be any type of beer. Preferred beer types comprise ales, stouts, porters, lagers, bitters, malt liquors, happoushu, high-alcohol beer, low-alcohol beer, low-calorie beer or light beer.

The term “granular starch” means raw uncooked starch, i.e. starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called gelatinization begins.

The term “initial gelatinization temperature” means the lowest temperature at which gelatinization of the starch commences. Starch heated in water begins to gelatinize between 50° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this invention the initial gelatinization temperature of a given starch is the temperature at which birefringence is lost in 5% of the starch granules using the method described by Gorinstein. S, and Lii. C., Starch/Stärke, Vol. 44 (12) pp. 461-466 (1992).

The polypeptide “homology” means the degree of identity between two amino acid sequences. The homology may suitably be determined by computer programs known in the art, such as, GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-453. The following settings for polypeptide sequence comparison are used: GAP creation penalty of 3.0 and GAP extension penalty of 0.1.

The term “acid alpha-amylase” means an alpha-amylase activity (E.C. 3.2.1.1) which added in an effective amount has activity at a pH in the range of 3.0 to 7.0, preferably from 3.5 to 6.0, or more preferably from 4.0-5.0.

### Enzymes

Acid alpha-amylases comprising a CBM: Acid alpha-amylases comprising a CBM are polypeptides within EC 3.2.1.1 having acid alpha-amylase activity and comprising a carbohydrate binding module, preferably the CBM is a starch binding domain (SBD), and preferably of family CBM20. The acid alpha-amylases comprising a CBM to be used in the present invention may be a hybrid enzyme or the polypeptide may be a wild type enzyme which already comprises a catalytic module having alpha-amylase activity and a carbohydrate-binding module. The acid alpha-amylases comprising a CBM to be used in the process of the invention may also be a variant of such a wild type enzyme. The hybrid may be produced by fusion of a first DNA sequence encoding a first amino acid sequences and a second DNA sequence encoding a second amino acid sequences, or the hybrid may be produced as a completely synthetic gene based on knowledge of the amino acid sequences of suitable CBMs, linkers and catalytic domains. The term “hybrid enzyme” is used herein to characterize polypeptides which are acid alpha-amylases comprising a CBM, which polypeptides comprises a first amino acid sequence comprising a catalytic module having alpha-amylase activity and a second amino acid sequence comprising at least one carbohydrate-binding module wherein the first and the second are derived from different sources. The term “source” being understood as e.g. but not limited to a parent polypeptide, e.g. an enzyme, e.g. an amylase or glucoamylase, or other catalytic activity comprising a suitable catalytic module and/or a suitable CBM and/or a suitable linker. The parent polypeptides of the CBM and the acid alpha-amylase activity may be derived from the same strain, and/or from the same species or it may be derived from different stains of the same species or from strains of different species. Both fungal and bacterial parent polypeptides are preferred as well as fungal and bacterial wild types and variants of wild types.

Preferred for the invention is any acid alpha-amylase comprising a CBM including but not limited to the fungal derived hybrid enzymes and wild type variants disclosed in PCT/US2004/020499 [NZ10490], and in Danish Patent application [NZ10729] filed on the same day as the present application as well as bacterial derived hybrids, wild types or wild type variants disclosed in Danish Patent application [NZ10753] filed on the same day as the present application More preferred is an enzyme having acid alpha-amylase activity and comprising a CBM which enzyme has the amino acid sequence disclosed as SEQ ID NO:1 (JA001) comprising a catalytic domain identical to the A. niger acid alpha-amylase and a CBM identical to the A. kawachii alpha-amylase CBM, the sequence disclosed as SEQ ID NO:2 (JA126) or the sequence disclosed as SEQ ID NO:3 (JA129) or acid alpha-amylases comprising a CBM which acid alpha-amylases has an amino acid sequence having at least 50%, 60%, 70%, 80%, 85% 90% or even at least 95% identity to any of the aforementioned amino acid sequences.

CBM-containing hybrid enzymes, as well as detailed descriptions of the preparation and purification thereof, are known in the art [see, e.g. WO 90/00609, WO 94/24158 and WO 95/16782, as well as Greenwood et al. Biotechnology and Bioengineering 44 (1994) pp. 1295-1305].

Alpha-amylase not comprising a CBM: Also alpha-amylase not comprising a CBM may be present during the process of the invention, e.g. as a fungal acid alpha-amylase such as the acid fungal alpha-amylase derived from Aspergillus niger and/or as a bacterial alpha-amylase, e.g. an alpha-amylases derived from Bacillus sp.

Alpha-amylase not comprising a CBM may be obtained as Mycolase from DSM (Gist Brochades), BAN™, TERMAMYL™ SC, FUNGAMYL™, LIQUOZYME™ X and SAN™ SUPER, SAN™ EXTRA L (Novozymes A/S) and Clarase L-40,000, DEX-LO™, Spezyme FRED, SPEZYME™ M, and SPEZYME™ DELTA AA (Genencor Int.).

Beta-glucanase (E.C. 3.2.1.4): The process of the invention may be carried out in the presence of an effective amount of a suitable beta-glucanase. The beta-glucanase may be of microbial origin, such as derivable from a strain of a bacteria (e.g. Bacillus) or from a filamentous fungus (e.g., Aspergillus, Trichoderma, Humicola, Fusarium). Preferred are beta-glucanases derived from Trichoderma sp., such as the beta-glucanase having the sequence shown in SEQ ID NO:8 (WO200014206), preferably T. reesei such as the beta-glucanase having the sequence shown in SEQ ID NO:6 or T. viride such as the beta-glucanase having the sequence shown in SEQ ID NO:7.

A beta-glucanases to be used in the processes of the invention may be an endo-glucanase, such as an endo-1,4-beta-glucanase. Commercially available beta-glucanase preparations which may be used include CELLUCLAST®), CELLUZYME®, CEREFLO® and ULTRAFLO® (available from Novozymes A/S), GC 880, LAMINEX™ and SPEZYME® CP (available from Genencor Int.) and ROHAMENT® 7069 W (available from Röhm, Germany). Preferred is CEREFLO®.

Beta-glucanases may be added in amounts of 0.01-5000 BGU/kg dry solids, preferably in the amounts of 0.1-500 BGU/kg dry solids, and most preferably from 1-50 BGU/kg dry solids and in the liquefaction step (down stream mash) in the amounts of 1.0-5000 BGU/kg dry solids, and most preferably from 10-500 BGU/kg dry solids.

Xylanase (EC 3.2.1.8 and other): The process of the invention may be carried out in the presence of an effective amount of a suitable xylanase which may be derived from a variety of organisms, including fungal and bacterial organisms, such as Aspergillus, Disporotrichum, Penicillium, Neurospora, Fusarium and Trichoderma.

Examples of suitable xylanases include xylanases derived from H. insolens (WO 92/17573; Aspergillus tubigensis (WO 92/01793); A. niger (Shei et al., 1985, Biotech. and Bioeng. Vol. XXVII, pp. 533-538, and Fournier et al., 1985, Bio-tech. Bioeng. Vol. XXVII, pp. 539-546; WO 91/19782 and EP 463 706); A. aculeatus (WO 94/21785).

The xylanase may also be a 1,3-beta-D-xylan xylanohydrolase (EC. 3.2.1.32).

Preferably the xylanase is a family 10 xylanase, and more preferably the xylanase is derived from Aspergillus sp. In specific embodiments the xylanase is Xylanase II from Aspergillus aculeatus disclosed in WO 94/21785 and shown in SEQ ID NO:4 or the xylanase is a xylanase from Trichoderma reesei having the sequence shown in SEQ ID NO:5 (SWISSPROT Q9P973).

Contemplated commercially available compositions comprising xylanase include SHEARZYME® 200L, SHEARZYME® 500L, BIOFEED WHEAT®, and PULPZYME™ HC (from Novozymes) and GC 880, SPEZYME® CP (from Genencor Int).

Xylanases may be added in the amounts of 1.0-1000 FXU/kg dry solids, preferably from 5-500 FXU/kg dry solids, preferably from 5-100 FXU/kg dry solids and most preferably from 10-100 FXU/kg dry solids.

Glucoamylase: A glucoamylase (E.C.3.2.1.3) may be used in the processes. Preferred is glucoamylases of fungal origin such as Aspergillus glucoamylases, in particular A. niger G1 or G2 glucoamylase (Boel et al. (1984), EMBO J. 3 (5), p. 1097-1102). Also preferred are variants thereof, such as disclosed in WO92/00381 and WO00/04136; the A. awamori glucoamylase (WO84/02921), A. oryzae (Agric. Biol. Chem. (1991), 55 (4), p. 941-949), or variants or fragments thereof. Preferred glucoamylases include the glucoamylases derived from Aspergillus niger, such as a glucoamylase having 50%, 55%, 60%, 65%, 70%, 75%, 80%, 85% or even 90% homology to the amino acid sequence set forth in WO00/04136 and SEQ ID NO: 13. Also preferred are the glucoamylases derived from Aspergillus oryzae, such as a glucoamylase having 50%, 55%, 60%, 65%, 70%, 75%, 80%, 85% or even 90% homology to the amino acid sequence set forth in WO00/04136 SEQ ID NO:2.

Other glucoamylases include Talaromyces glucoamylases, in particular derived from Talaromyces emersonii (WO99/28448), Talaromyces leycettanus (U.S. Pat. No. Re. 32,153), Talaromyces duponti, Talaromyces thermophilus (U.S. Pat. No. 4,587,215), Clostridium, in particular C. thermoamylolyticum (EP135,138), and C. thermohydrosulfuricum (WO86/01831).

Commercially available compositions comprising glucoamylase include AMG 200 L; AMG 300 L; SAN™ SUPER, SAN EXTRA L and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Phytase: An additional enzyme that may be used in the process of the invention is a phytases. The phytase may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any specificity, e.g., be a 3-phytase (E.C. 3.1.3.8), a 6-phytase (E.C. 3.1.3.26) or a 5-phytase (no E.C. number).

Commercially available phytases preferred according to the invention include BIO-FEED PHYTASE™, PHYTASE NOVO™ CT or L (Novozymes A/S), or NATUPHOS™ NG 5000 (DSM).

Another enzyme of may be a debranching enzyme, such as an isoamylase (E.C. 3.2.1.68) or a pullulanases (E.C. 3.2.1.41). Isoamylase hydrolyses alpha-1,6-D-glucosidic branch linkages in amylopectin and beta-limit dextrins and can be distinguished from pullulanases by the inability of isoamylase to attack pullulan, and by the limited action on alpha-limit dextrins. Debranching enzyme may be added in effective amounts well known to the person skilled in the art.

In a first preferred embodiment, the invention provides a process for production of ethanol, comprising the steps of: (a) providing a slurry comprising water and granular starch, (b) incubating said slurry in the presence of i) an acid alpha-amylase comprising a CBM and ii) a fermenting organism, e.g. a yeast, at a temperature of between 30° C. and 35° C. to produce to produce a fermentation product, and, (c) optionally recovering the fermentation product, e.g. ethanol. The steps (a), (b), and (c) may be performed sequentially; however, the process may comprise additional steps not specified in this description which are performed prior to, between or after any of steps (a), (b), and (c). Preferably the temperature under step (b) is between 28° C. and 36° C., preferably from 29° C. and 35° C., more preferably from 30° C. and 34° C., such as around 32° C. and the slurry is held in contact with the i) an acid alpha-amylase comprising a CBM and ii) a fermenting organism, e.g a yeast, for a period of time sufficient to allow hydrolysis of the starch and fermentation of the released sugars during step (b), preferably for a period of 25 to 190 hours, preferably from 30 to 180 hours, more preferably from 40 to 170 hours, even more preferably from 50 to 160 hours, yet more preferably from 60 to 150 hours, even yet more preferably from 70 to 140 hours, and most preferably from 80 to 130 hours, such as 85 to 110 hours. In a further preferred embodiment also a xylanase and/or a beta-glucanase is present during step b).

In a preferred embodiment the slurry prior to step b) is incubated at a temperature from 0 to 30° C. below the initial gelatinization temperature, e.g. at a temperature from 0 to 20° C., preferably from 0 to 10, more preferably from 5 to 10° C. below the initial gelatinization temperature. Preferably the slurry prior to step b) is incubated at a temperature from 0 to 30° C. below the initial gelatinization temperature, such as at from 35° C. to 45° C., from 40° C. to 50° C., or from 45° C. to 55° C.

The acid alpha-amylase comprising a CBM is added in an effective amount, which is a concentration of acid alpha-amylase activity sufficient for its intended purpose of converting the granular starch in the starch slurry to dextrins. Preferably the acid alpha-amylase comprising a CBM is present in an amount of 10-10000 AFAU/kg of DS, in an amount of 500-2500 AFAU/kg of DS, or more preferably in an amount of 100-1000 AFAU/kg of DS, such as approximately 500 AFAU/kg DS. When measured in AAU units the acid alpha-amylase activity is preferably present in an amount of 5-500000 AAU/kg of DS, in an amount of 500-50000 AAU/kg of DS, or more preferably in an amount of 100-10000 AAU/kg of DS, such as 500-1000 AAU/kg DS.

The glucoamylases is added in an effective amount, which is a concentration of glucoamylase amylase sufficient for its intended purpose of degrading the dextrins resulting from the acid alpha-amylase treatment of the starch slurry. Preferably the glucoamylase activity is present in an amount of 20-200 AGU/kg of DS, preferably 100-1000 AGU/kg of DS, or more preferably in an amount of 200-400 AGU/kg of DS, such as 250 AGU/kg DS. When measured in AGI units the glucoamylase activity is preferably present in an amount of 10-100000 AGI/kg of DS, 50-50000 AGI/kg of DS, preferably 100-10000 AGI/kg of DS, or more preferably in an amount of 200-5000 AGI/kg of DS.

Preferably the activities of acid alpha-amylase activity and glucoamylase activity are are added to the slurry in a ratio of between 0.3 and 5.0 AFAU/AGU. More preferably the ratio between acid alpha-amylase activity and glucoamylase activity is at least 0.35, at least 0.40, at least 0.50, at least 0.60, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.85, or even at least 1.9 AFAU/AGU. However, the ratio between acid alpha-amylase activity and glucoamylase activity should preferably be less than 4.5, less than 4.0, less than 3.5, less than 3.0, less than 2.5, or even less than 2.25 AFAU/AGU. In AUU/AGI the activities of acid alpha-amylase and glucoamylase are preferably present in a ratio of between 0.4 and 6.5 AUU/AGI. More preferably the ratio between acid alpha-amylase activity and glucoamylase activity is at least 0.45, at least 0.50, at least 0.60, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, or even at least 2.5 AUU/AGI. However, the ratio between acid alpha-amylase activity and glucoamylase activity is preferably less than 6.0, less than 5.5, less than 4.5, less than 4.0, less than 3.5, or even less than 3.0 AUU/AGI.

In the first aspect of the invention the xylanase may preferably be present in amounts of 1-50000 FXU/kg DS, preferably 5-5000 FXU/kg DS, or more preferably 10-500 FXU/kg DS and the beta-glucanase may be present in the amounts of 0.01-500000 EGU/kg DS, preferably from 0.1-10000 EGU/kg DS, preferably from 1-5000 EGU/kg DS, more preferably from 10-500 EGU/kg DS and most preferably from 100-250 EGU/kg DS.

The enzyme activities may preferably be dosed in form of the composition of the second aspect of the invention, and preferably the composition comprises concentrations of the aforementioned enzymes in strength equal to 10 times, 100 times, 1000 times or even 10000 times the concentrations in the slurry.

In a preferred embodiment the starch slurry comprises water and 5-60% DS (dry solids) granular starch, preferably 10-50% DS granular starch, more preferably 15-40% DS, especially around 20-25% DS granular starch. The granular starch to be processed in the processes of the invention may in particular be obtained from tubers, roots, stems, cobs, legumes, cereals or whole grain. More specifically the granular starch may be obtained from corns, cobs, wheat, barley, rye, milo, sago, cassaya, tapioca, sorghum, rice, peas, bean, banana or potatoes. Preferred are both waxy and non-waxy types of corn and barley. Most preferred are cereals, especially wheat, barley and/or rye. The granular starch to be processed may preferably be derived from milled whole grain. The raw material comprising the granular starch is preferably milled in order to open up the structure and allowing for further processing. The granular starch is preferably dry milled grain, e.g. wheat, barley and/or rye. As wheat, barley and/or rye comprises significant amounts of beta-glucan and xylan a dry milled grist comprising these grain species can result in a high mash viscosity in a conventional process comprising liquefaction and/or saccharification of ungelatinized granular starch. Thus granular starch derived from dry milled wheat, barley and/or rye are particularly preferred for the process of the invention.

After being subjected to the process of the first aspect of the invention at least 85%, 86%, 87%, 88%, 89%, 90%, 91%, 92%, 93%, 94%, 95%, 96%, 97%, 98%, or preferably 99% of the dry solids of the granular starch is converted into ethanol.

The fermented slurry comprises at least 7%, at least 8%, at least 9%, at least 10% such as at least 11%, at least 12%, at least 13%, at least 14%, at least 15% such as at least 16% ethanol.

The ethanol may optionally be recovered following fermentation. The ethanol recovery may be performed by any conventional manner such as e.g. distillation and may be used as fuel ethanol and/or potable ethanol and/or industrial ethanol.

### Materials and Methods

### Acid Alpha-Amylase Activity

When used according to the present invention the activity of any acid alpha-amylase activity may be measured in AFAU (Acid Fungal Alpha-amylase Units). Alternatively activity of acid alpha-amylase may be measured in AAU (Acid Alpha-amylase Units).

### Acid Alpha-Amylase Units (MU)

The acid alpha-amylase activity can be measured in AAU (Acid Alpha-amylase Units), which is an absolute method. One Acid Amylase Unit (MU) is the quantity of enzyme converting 1 g of starch (100% of dry solids) per hour under standardized conditions into a product having a transmission at 620 nm after reaction with an iodine solution of known strength equal to the one of a color reference.

### Standard Conditions/Reaction Conditions:

| | |
|---|---|
| Substrate: | Soluble starch. Concentration |
| | approx. 20 g DS/L. |
| Buffer: | Citrate, approx. 0.13 M, pH = 4.2 |
| Iodine solution: | 40.176 g potassium iodide + 0.088 g iodine/L |
| City water | 15°-20° dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as calorimetric indicator. Lintner starch is obtained by dilute hydrochloric acid treatment of native starch so that it retains the ability to color blue with iodine. Further details can be found in EP0140410B2, which disclosure is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 FAU is defined as the amount of enzyme which degrades 5.260 mg starch dry solids per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

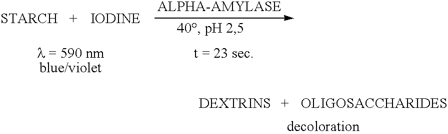

Standard Conditions/Reaction Conditions:

| | |
|---|---|
| Substrate: | Soluble starch, approx. 0.17 g/L |
| Buffer: | Citrate, approx. 0.03 M |
| Iodine (I2): | 0.03 g/L |
| CaCl2: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

A folder EB-SM-0259.02/01 describing this analytical method in more detail is available upon request to Novozymes A/S, Denmark, which folder is hereby included by reference.

### Bacterial Alpha-Amylase Activity (KNU)

The bacterial alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Glucoamylase Activity

Glucoamylase activity may be measured in AGI units or in AmyloGlucosidase Units (AGU)

### Glucoamylase Activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”. Vol. 1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

### Standard Conditions/Reaction Conditions:

| | |
|---|---|
| Substrate: | Soluble starch, concentration |
| | approx. 16 g dry solids/L. |
| Buffer: | Acetate, approx. 0.04 M, pH = 4.3 |
| pH: | 4.3 |
| Incubation temperature: | 60° C. |
| Reaction time: | 15 minutes |
| Termination of the reaction: | NaOH to a concentration of |
| | approximately 0.2 g/L (pH~9) |
| Enzyme concentration: | 0.15-0.55 AAU/mL. |

The starch should be Lintner starch, which is a thin-boiling starch used in the laboratory as calorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG Incubation:

| | |
|---|---|
| Substrate: | maltose 23.2 mM |
| Buffer: | Acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

Color Reaction:

| | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Xylanolytic Activity

The xylanolytic activity can be expressed in FXU-units, determined at pH 6.0 with remazol-xylan (4-O-methyl-D-glucurono-D-xylan dyed with Remazol Brilliant Blue R, Fluka) as substrate.

A xylanase sample is incubated with the remazol-xylan substrate. The background of non-degraded dyed substrate is precipitated by ethanol. The remaining blue color in the supernatant (as determined spectrophotometrically at 585 nm) is proportional to the xylanase activity, and the xylanase units are then determined relatively to an enzyme standard at standard reaction conditions, i.e. at 50.0° C., pH 6.0, and 30 minutes reaction time.

A folder EB-SM-352.02/01 describing this analytical method in more detail is available upon request to Novozymes A/S, Denmark, which folder is hereby included by reference.

### Cellulytic Activity

The cellulytic activity may be measured in endo-glucanase units (EGU), determined at pH 6.0 with carboxymethyl cellulose (CMC) as substrate. A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes. One EGU is defined as the amount of enzyme that reduces the viscosity to one half under these conditions. The amount of enzyme sample should be adjusted to provide 0.01-0.02 EGU/ml in the reaction mixture. The arch standard is defined as 880 EGU/g.

A folder EB-SM-0275.02/01 describing this analytical method in more detail is available upon request to Novozymes A/S, Denmark, which folder is hereby included by reference.

### Enzymes Preparation

The following enzyme preparations were used:

Bacterial alpha-amylase; An enzyme preparations comprising a polypeptide with alpha-amylase activity (E.C. 3.2.1.1) derived from B. stearothermophilus and having the amino acid sequence disclosed as SEQ. NO:4 in WO99/194671. Activity: 120 KNU/g (density=1.20-1.25 g/mL).

A composition comprising an acid fungal alpha-amylase comprising a CBM having the sequence shown in SEQ ID NO:1 and some glucoamylase. Activities: 329 AFAU/g, 31 AGU/g (density=1.2 g/mL).
A glucoamylase composition |derived from Aspergillus niger comprising glucoamylase and some acid fungal alpha-amylase|. Activity: 363 AGU/g, 86 AFAU/g (density=1.2 g/mL).
A glucoamylase composition |derived from a genetically modified Aspergillus niger microorganism (Spirizyme Fuel) comprising glucoamylase and some acid fungal alpha-amylase|. Activity: 750 AGU/g, 30 AFAU/g.
A glucoamylase composition derived from a genetically modified Aspergillus niger microorganism (Spirizyme Plus) comprising glucoamylase and some acid fungal alpha-amylase|. Activity: 363 AGU/g, 86 AFAU/g.
|An enzyme composition (Novozym 50024) comprising xylanase and cellulase activities derived from respectively Trichoderma reesei and Aspergillus aculeatus. |Activity: 300 FXU/g+350 EGU/g (density=1.2 g/mL).
Yeast: Dried baker's yeast from De Danske Spriffabrikker A/S (Danish Distillers)

### EXAMPLE 1

This example illustrates a process of the invention using an acid alpha-amylase comprising a CBM. A 20% D.S. slurry of milled wheat was made in RT tab water. For each treatment 2×250 g slurry was portioned in 500 mL blue cap flasks. The pH was adjusted to 4.5 using 6 N HCl. Enzyme activities were dosed according to table 6, and the flasks were incubated for one hour at 55° C. in a shaking water bath. The flasks were cooled to 32° C. and 0.25 g dry bakers yeast added. The flasks were placed in a water bath at 32° C. for 72 hours. Weight loss data was recorded. At 50 and 72.5 hours the flasks were weighed and CO₂ weight loss measured for monitoring of the fermentation progress. The relationship used between amount of CO₂ loss and the weight of ethanol was: CO₂ loss (g)×1.045=EtOH (g).

TABLE 1
Wheat; the weight loss (g) at 50 hours and at 72 hours.

| | | | | |
|---|---|---|---|---|
| AGU/kg DS | 0.3 | 0.3 | 0.3 | 0.3 |
| AFAU/kg DS | 0 | 0.05 | 0.17 | 0.3 |
| Weight loss (g), 50 hours | 10.98 | 12.98 | 14.37 | 15.18 |
| Weight loss (g), 72.5 hours | 12.90 | 15.49 | 16.69 | 17.79 |
| Ethanol % w/w, 50 Hrs* | 4.80 | 5.73 | 6.37 | 6.76 |
| Ethanol % w/w, 72 Hrs* | 5.69 | 6.90 | 7.48 | 8.01 |
| Ethanol % w/w, 100 hrs** | 6.26 | 7.57 | 7.82 | 8.25 |

Glucoamylase (AGU), Acid alpha-amylase comprising a CBM (AFAU)
*based on weight loss at 50 and 72 hours, CO₂ loss (g) × 1.045 = EtOH (g),
**based on HPLC at 100 hours.

### EXAMPLE 2

A conventional ethanol process using a traditional pre-liquefaction called the non-pressure cooking (NPC) is compared with the process of the invention. Traditional non-pressure batch cooking processes for production of potable alcohol is described in the Novozymes publication No. 2001-10782-01 entitled “Use of Novozymes enzymes in alcohol production”.

A 20% D.S. slurry of the milled barley grain was made in room temperature (RT) tap water. The viscosity was measured using a viscometer type HAAKE Viscotester VT02.

The NPC pre-treatment of the conventional ethanol process was performed in 6×1-litre tubs with stirring. Bacterial alpha-amylase was added and the tubs were placed in water bath at 65° C. When the temperature in the mash reached 55° C. the heating was increased to heat the mash to 90° C. over 60 minutes. The temperature was then adjusted to 32° C. and 3×250 g mash was portioned in 500 mL blue cap flasks with air locks. To all flasks 0.25 g dry bakers yeast was added (corresponding to 5-10 million vital cells/g mash). Enzyme activities were added according to the table below and each flask was weighed. The flasks were placed in a shaking water bath at 32° C. At 72 hours the flasks were weighed and CO₂ weight loss measured for monitoring of the fermentation progress. The relationship used between amount of CO₂ loss and the weight of ethanol was: CO₂ loss (g)×1.045=EtOH (g).

For the process of the invention a 30% D.S. slurry of the milled barley was made in room temperature (RT) tap water. 500 mL blue cap fermentation flasks each with 250 g slurry was fitted with air locks. Using 4.0 molar H₂SO₄ the pH was adjusted to 5.0 and the viscosity was measured using a viscometer type HAAKE Viscotester VT02. Xylanase, cellulase, glucoamylase and an acid alpha-amylase comprising a CBM was dosed according to table 2. The temperature was then adjusted to 32° C. and the flasks were held under magnet stirring and fermentation was monitored as described above using 0.25 g dry baker's yeast (corresponding to 5-10 million vital cells/g mash at beginning of the fermentation). Time for the invention was counted from the inoculation.

TABLE 2
Barley, the weight loss (g) at 72 hours and at 91 hours.

| | Traditional non- | Process of the |
|---|---|---|
| | pressure cooking | invention |
| % DS | 20 | 30 |
| Viscosity before fermentation, cP | 2000 | 8000 |
| FXU/kg DS | 0 | 300 |
| EGU/kg DS | 0 | 350 |
| KNU/kg DS | 36 | 0 |
| AFAU/kg DS | 0 | 280 |
| AGU/kg DS | 163 | 470 |
| AFAU/AGU | 0 | 0.60 |
| Weight loss (g), 72 hours | 12.2 | 15 |
| Weight loss (g), 91 hours | 14.0 | 16.7 |
| Final viscosity cP | <30 | <30 |
| Ethanol % w/w, 72 Hrs | 5.1 | 6.7 |
| Ethanol % w/w, 91 Hrs | 6.2 | 7.5 |

Bacterial alpha-amylase acid (KNU), acid alpha-amylase comprising a CBM (AFAU) and glucoamylase (AGU) activity was added according to the table.

### EXAMPLE 3

This example illustrates the process of the invention using various raw materials. A 30% D.S. slurry of the milled cereal was made in RT tab water. For each treatment 2×250 g was portioned in 500 mL blue cap flasks. The temperature was then adjusted to 32° C. and fermentation was performed and monitored as described above using 0.25 g dry baker's yeast (corresponding to 5-10 million vital cells/g mash at the beginning of the fermentation). Time is counted from the inoculation.

Using 4.0 molar H₂SO₄ the pH was adjusted to 5.0 and the viscosity was measured using a viscometer type HAAKE Viscotester VT02. Xylanase, cellulase, glucoamylase and acid alpha-amylase were dosed according to table 3. The flasks were adjusted to 32° C. and 0.25 g dry bakers yeast added. The flasks were kept under magnet stirring in a water bath at 32° C. for 91 hours.

TABLE 3
Wheat and Rye, the weight loss (g) at 91 hours.

Cereal

| | Wheat | Rye |
|---|---|---|
| % DS | 30 | 30 |
| Viscosity before fermentation, cP | 950 | 16500 |
| FXU/kg DS | 300 | 300 |
| EGU/kg DS | 350 | 350 |
| AFAU/kg DS | 280 | 280 |
| AGU/kg DS | 470 | 470 |
| AFAU/AGU | 0.60 | 0.60 |
| Weight loss (g), 91 hours | 17.2 | 17.1 |
| Viscosity after fermentation, cP | <30 | <30 |
| Ethanol w/w %, 91 Hrs | 7.7 | 7.7 |

Acid alpha-amylase comprising a CBM (AFAU), and glucoamylase (AGU) activity was added according to the table.
* based on weight loss at 91 hours, CO₂ loss (g) × 1.045 = EtOH (g).

1-24. (canceled)

25. A process comprising the steps of:
(a) providing a slurry comprising water and granular starch
(b) holding said slurry in the presence of i) an acid alpha-amylase comprising a carbohydrate binding module, and/or ii) a fermenting organism, to produce a fermentation product, and
(c) optionally recovering the fermentation product.

26. The process of claim 25, wherein the fermentation organism is yeast.

27. The process of claim 25, wherein step b further comprises the presence of iii) a xylanase and iv) a beta-glucanase.

28. The process of claim 25, wherein step b further comprises the presence of a glucoamylase.

29. The process of claim 25, wherein the slurry prior to step b) is incubated at a temperature from 0 to 30° C. below the initial gelatinization temperature.

30. The process of claim 25, wherein the fermentation product is fuel ethanol, potable ethanol and/or industrial ethanol.

31. The process of claim 25, wherein the fermented slurry comprises at least 7% ethanol.

32. The process of claim 25, wherein the temperature under step (b) is between 28° C. and 36° C.

33. The process of claim 25, wherein the acid alpha-amylase comprising a CBM is an alpha-amylase comprising an amino acid sequence which has at least 70% homology to any amino acid sequence selected from the group consisting of SEQ ID NO: 1, SEQ ID NO: 2 and SEQ ID NO: 3.

34. The method of claim 27, wherein the xylanase is derived from a strain of Aspergillus sp.

35. The method of claim 27, wherein the beta-glucanase is derived from a strain of Trichoderma sp.

36. The process of claim 25, wherein the acid alpha-amylase activity is present in an amount of 50-500 AFAU/kg of DS.

37. The process of claim 28, wherein the glucoamylase activity is present in an amount of 20-200 AGU/kg of DS.

38. The process of claim 25, wherein the starch slurry has 5-60% DS granular starch.

39. The process of claim 25; wherein the ethanol content during step b reaches at least 7% (w/w).

40. The process of claim 25, wherein the pH during step (b) is in the range of 3.0 to 7.0.

41. The process of claim 25, wherein the granular starch is obtained from tubers, roots, stems, fruits, seeds, cereals and/or whole grain.

42. The process of claim 25, wherein the granular starch is obtained from cereals.

43. The process of claim 25, wherein the granular starch is obtained from corn.

44. A composition comprising a) an acid alpha-amylase comprising a CBM and b) a glucoamylase and/or c) a beta-glucanase, and/or d) a xylanase.
